SEQUENCE LISTING (19 sequences)

SEQ ID NO: 1 - protein, 462 aa, Artificial Sequence
  H27 Humanized antibody construct comprising sequences from mus musculus and homo sapiens
MGWSCIILFLVATATGVHSQVQLVQSGAEVKKPGASVKVSCKASGYTFTSYWMHWVKQRPGQGLEWIGNINPSNGGTNYNEKFKSKATLTVDKSTSTAYMELSSLRSEDTAVYYCELMQGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 462 aa, Artificial Sequence
  H28 Humanized antibody construct comprising sequences from mus musculus and homo sapiens
MGWSCIILFLVATATGVHSQVQLVQSGAEVKKPGASVKVSCKASGYTFTSYWMHWVRQAPGQGLEWIGNINPSNGGTNYNEKFKSKATMTRDTSTSTAYMELSSLRSEDTAVYYCELMQGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 238 aa, Artificial Sequence
  L13 Humanized antibody construct comprising sequences from mus musculus and homo sapiens
MGWSCIILFLVATATGVHSDIVMTQSPLSLPVTLGQPASISCRSSKSLLYKDGKTYLNWFQQRPGQSPQLLIYLMSTRASGVPDRFSGGGSGTDFTLKISRVEAGDVGVYYCQQLVEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 238 aa, Artificial Sequence
  L16 Humanized antibody construct comprising sequences from mus musculus and homo sapiens
MGWSCIILFLVATATGVHSDIVMTQSPLSNPVTLGQPVSISCRSSKSLLYKDGKTYLNWFLQRPGQSPQLLIYLMSTRASGVPDRFSGGGSGTDFTLKISRVEAEDVGVYYCQQLVEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 439 aa, Homo sapiens
  Amino acids 586-785 of human NogoA
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKELERPHRD

SEQ ID NO: 6 - protein, 12 aa, Homo sapiens
VLPDIVMEAPLN

SEQ ID NO: 7 - protein, 16 aa, Homo sapiens
TPSPVLPDIVMEAPLN

SEQ ID NO: 8 - protein, 16 aa, Homo sapiens
VLPDIVMEAPLNSAVP

SEQ ID NO: 9 - protein, 16 aa, Homo sapiens
YESIKHEPENPPPYEE

SEQ ID NO: 10 - protein, 1192 aa, Homo sapiens
  Human NogoA
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 11 - protein, 113 aa, Artificial Sequence
  H20 Vh humanized antibody construct comprising sequences from mus musculus and homo sapiens
QVQLVQSGAEVKKPGASVKVSCKASGYTFTSYWMHWVRQAPGQGLEWIGNINPSNGGTNYNEKFKSKATMTRDTSTSTAYMELSSLRSEDTAVYYCELGQGYWGQGTLVTVSS

SEQ ID NO: 12 - protein, 113 aa, Artificial Sequence
  H27 Vh humanized antibody construct comprising sequences from mus musculus and homo sapiens
QVQLVQSGAEVKKPGASVKVSCKASGYTFTSYWMHWVKQRPGQGLEWIGNINPSNGGTNYNEKFKSKATLTVDKSTSTAYMELSSLRSEDTAVYYCELMQGYWGQGTLVTVSS

SEQ ID NO: 13 - protein, 113 aa, Artificial Sequence [sequence omitted]

SEQ ID NO: 14 - protein, 112 aa, Artificial Sequence
  L13 Vl humanized antibody construct comprising sequences from mus musculus and homo sapiens
DIVMTQSPLSLPVTLGQPASISCRSSKSLLYKDGKTYLNWFQQRPGQSPQLLIYLMSTRASGVPDRFSGGGSGTDFTLKISRVEAEDVGVYYCQQLVEYPLTFGQGTKLEIK

SEQ ID NO: 15 - protein, 112 aa, Artificial Sequence [sequence omitted]

SEQ ID NO: 16 - protein, 220 aa, Mus musculus
MDFGLIFFIVGLLKGVQCEVKLLESGGLVQPGGSLKLSCVVSGFDFRRNWMSWVRQAPGKGLEWIGEINPDSSKINYTPSLKDKFIISRDNAKNTLYLQVSTVRSEDTALYTCVRPVWMYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVA

SEQ ID NO: 17 - protein, 237 aa, Mus musculus
MSPAQFLFLLVLWIRETSGDVLLTQTPLTLSITIGQPASISCKSSQSLLHSDGKTYLNWLLQRPGQSPKRLIYLVSKLDSGVPDEFTGSGSGTDFTLKISRVEAGDLGLYYCWQGTHFPQTFGGGTKLEIKRADAAPTVSIFPPSSGQLTSGGASVVCFLNNFYPKDINVKWKIDGSERQNGVLNSWDQDSKDSTYSMSSTLTLTKDEYERHNSYTCEATHKTSTSPIVKSFNRGEC

SEQ ID NO: 18 - protein, 246 aa, Mus musculus
MEFGLSWVFLVAILEGVQCEVQLVESGGGLVQPGGSLRLSCAASGFTFSNYWMSWVRQAPGKGLEWVATIKQDGSQKNYVDSVKGRFTISRDNAKNSLYLRLNSLRAEDTAVYYCATELFDLWGRGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 19 - protein, 234 aa, Mus musculus
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2011/0268729 A1
(43) Pub. Date: Nov. 3, 2011

(54) TREATMENT OF AMYOTROPHIC LATERAL SCLEROSIS BY NOGO-A-ANTAGONIST

(76) Inventors: Bams Abila, Hertfordshire (GB); Sean Matthew Cleveland, Hertfordshire (GB); Paul Andrew Hamblin, Hertfordshire (GB); Rabinder Kumar Prinjha, Hertfordshire (GB)

(21) Appl. No.: 13/003,657

(22) PCT Filed: Jul. 10, 2009
(86) PCT No.: PCT/EP2009/058832

§ 371 Date: Jan. 11, 2011

Related U.S. Application Data
(62) Division of application No. 61/079,874, filed on Jul. 11, 2008.

(51) Int. Cl. A61K 39/395; C07K 16/18; A61P 25/00; A61K 31/428

(52) U.S. Cl. 424/133.1; 514/367; 424/139.1; 530/387.9

(57) ABSTRACT

The invention relates to methods for the treatment or prophylaxis of amyotrophic lateral sclerosis, comprising administering to a patient in need thereof a therapeutically effective amount of a Nogo-A antagonist.

### FIELD OF THE INVENTION

The present invention relates to the treatment or prophylaxis of amyotrophic lateral sclerosis and other neurodegenerative diseases. More particularly, the invention relates to the use of an anti-Nogo-A antibody in the treatment or prophylaxis of amyotrophic lateral sclerosis.

### BACKGROUND

Amyotrophic lateral sclerosis (ALS), also known as Lou Gehrig's Disease or Maladie de Charcot, is the most common adult-onset motor neuron disease. The primary disease hallmark is the progressive degeneration of the upper and lower motor neurons in the corticospinal tracts. Dysfunction of lower motor neurons (in the brainstem and spinal cord) triggers generalized weakness, muscle atrophy and paralysis. Failure of the respiratory muscles is generally the fatal event, occurring within 1-5 years of onset.

ALS is the most common motor neuron disease in adults affecting approximately 30,000 people in the United States and 5,000 in the United Kingdom each year (Leigh & Swash, 1991). The typical age of onset is between 50 and 70 years, although sometimes occurring at a younger age. Most cases (90-95%) are classified as sporadic ALS (sALS) and the remainder are inherited and referred to as familial ALS (fALS). Sporadic and familial forms are clinically and pathologically similar, suggesting a common pathogenesis (Bruijn et al, 2004). However, the precise cause for most cases is still unknown, and there is no effective remedy to stop the course of the disease. The treatment and prophylaxis of ALS remains a significant unmet medical need.

### SUMMARY OF THE INVENTION

The present invention provides a method for the treatment or prophylaxis of ALS, comprising administering to a patient in need thereof a therapeutically effective amount of a Nogo-A antagonist.

The Nogo-A antagonist may be a neutralising anti-Nogo-A antibody or a fragment thereof, such as murine antibodies 2A10 and 2C4 (described in WO2005016544, the content of which is incorporated herein by reference in its entirety). Typically the anti-Nogo-A antibody will be a humanised antibody such as a humanised variant of 2A10, for example H20L16, H28L16, H28L13 and H27L16 (as described in WO2007/068750, the content of which is incorporated herein by reference in its entirety), a human antibody, or a fragment thereof. Preferably the antibody is H28L16. Amino acid sequences of the humanised constructs of the heavy chain and light chain variable region of 2A10 are presented as SEQ ID NOs: 11 to 15 herein. Full length heavy and light chain humanised variants of 2A10 are presented as SEQ ID NOs: 1 to 4.

The anti-Nogo-A antibody may also be any of the antibodies described in WO2004/052932, the content of which is incorporated herein by reference in its entirety. Examples of antibodies disclosed in WO2004/052932 are 11C7, including humanised variants thereof. The sequence of the variable regions of 11C7 is shown in SEQ ID NOs: 16 and 17. Human anti-Nogo-A antibodies are also described in WO2005/028508 and in WO2009/056509, the contents of which are incorporated herein by reference in their entirety. Specific antibodies disclosed in WO2009/056509 include the human antibody 6A3, having variable regions as shown in SEQ ID NOs: 18 and 19.

The Nogo-A antibody may comprise heavy chains of SEQ ID NO: 1 or 2, and light chains of SEQ ID NO: 3 or 4. In an embodiment, the Nogo-A antibody or fragment thereof comprises one or more, optionally six, of the CDRs of 2A10, H28L16 or 6A3. In an embodiment, the Nogo-A antibody or fragment thereof is an antibody that binds to the same human Nogo-A epitope as H28L16 (human Nogo-A 610-621aa, which includes VLPDIVMEAPLN (SEQ ID NO:6) or competes with the binding of H28L16 to human Nogo-A.

Human Nogo-A can be described by an amino acid sequence as set forth in SEQ ID NO:10 below.

In an embodiment, the Nogo-A antagonist is administered with a compound having anti-glutamate activity. In a specific embodiment, the compound having anti-glutamate activity is riluzole. In another embodiment, the compound having anti-glutamate activity is an antagonist of an AMPA receptor, such as a 2,3-benzodiazepine compound, in particular, talampanel. In another embodiment, the compound having anti-glutamate activity is TRO19622 or ceftriaxone. The Nogo-A antagonist and the compound having anti-glutamate activity may be administered to the patient simultaneously, sequentially or separately. Where the compound having anti-glutamate activity is riluzole, about 50 mg to about 150 or 200 mg riluzole may be administered to the patient daily, typically 100 mg riluzole is administered to the patient daily. Riluzole is typically orally administered. Where the compound having anti-glutamate activity is Talampanel, Talampanel is administered, typically orally, at about 10 mg to about 250 mg, from once to five times per day. In one embodiment, Talampanel is administered at a dosage of 25 mg or 50 mg, from once to five times per day, optionally three times per day.

The Nogo-A antagonist may be administered in an amount of from 0.1 mg/kg to 300 mg/kg. Usually from about 2 mg/kg to about 40 mg/kg of Nogo-A antagonist is administered to the patient, typically by the intravenous route. In an embodiment, the Nogo-A antagonist is administered subcutaneously. The Nogo-A antagonist is generally administered to the patient weekly, once every two weeks, or once every four weeks.

In another embodiment, the invention provides a method for the treatment or prophylaxis of ALS in subjects who have shown an inadequate response to therapy, or are refractory to therapy, with a compound having anti-glutamate activity. The compound having anti-glutamate activity is typically riluzole.

In another embodiment, the invention provides a Nogo-A antagonist for the treatment or prophylaxis of ALS.

In another embodiment, the invention provides the use of a Nogo-A antagonist in the manufacture of a medicament for the treatment or prophylaxis of ALS. The invention also provides pharmaceutical compositions comprising at least one Nogo-A antibody, and a kit of parts comprising at least one Nogo-A antibody and instructions for use of said antibody in the treatment of at least one disease of the invention (where the disease is ALS or MS, the instructions may include instruction to co-administer the Nogo-A antibody with a compound having anti-glutamate activity). The Nogo-A antibody may be selected from the group of: H28L16 (SEQ ID NO:2 and SEQ ID NO:4), H28L13 (SEQ ID NO:2 and SEQ ID NO:3) and H27L16 (SEQ ID NO:1 and SEQ ID NO:4). The present invention also provides pharmaceutical compositions comprising at least on Nogo-A antibody and at least one compound having anti-glutamate activity. In some instances, the compound have anti-glutamate activity is riluzole.

Moreover, the evidence contained herein suggests that Nogo-A antagonism may also serve a therapeutic purpose in other muscle diseases in which Nogo-A has been shown to be upregulated in muscle biopsies. Such diseases include, but are not limited to, inclusion body myositis (IBM), polymyositis, dermatomyositis, morphologically nonspecific myopathies (Wojcik et al (2007) Acta Neuropathol 114(5) 517-526) and also cardiac muscle diseases including heart failure, particularly congestive heart failure (T A Bullard, 2007). Indeed, the evidence herein suggests that the use of Nogo-A antagonism could extend to all muscle diseases caused by or associated with denervation.

The ability of systemic anti-Nogo-A treatment to result in significant neuroprotection in the CNS is further consistent with its therapeutic use in a wide range of neurological diseases including, but not limited to, Alzheimer's disease, Parkinson's disease, stroke, multiple-sclerosis, neuropathic pain and other diseases involving Nogo-A expression upregulation or Nogo-A mediated inhibition of regeneration or neuronal survival.

Accordingly, in another embodiment, the present invention provides a method for the treatment or prophylaxis of diseases in which Nogo-A expression is upregulated, such as muscle diseases including inclusion body myositis, polymyositis, dermatomyositis, morphologically nonspecific myopathies and (congestive) heart failure, or neurological diseases and disorders including Alzheimer's disease, Parkinson's disease, stroke, multiple-sclerosis, neuropathic pain, comprising administering to a patient in need thereof a therapeutically effective amount of an Nogo-A antagonist. The Nogo-A antagonist may be an anti-Nogo-A antibody, such as H28L16 (SEQ ID NO:2 and SEQ ID NO:4) or 6A3 (with a variable heavy and light chain as set out in SEQ ID NO:18 and SEQ ID NO:19).

Glutamate antagonism has also been proposed for the therapy of multiple sclerosis (Killestein et al. J. Neurol. Sci. 15 Jun. 2005, Pages 113-115). In another embodiment, therefore, the present invention provides a method for the treatment or prophylaxis of multiple sclerosis, particularly primary progressive MS, comprising administering to a patient in need thereof a therapeutically effective amount of an Nogo-A antagonist and a compound having anti-glutamate activity. The Nogo-A antagonist may be an anti-Nogo-A antibody, such as H28L16 (SEQ ID NO:2 and SEQ ID NO:4) or 6A3 (with a variable heavy and light chain as set out in SEQ ID NO:18 and SEQ ID NO:19).

### BRIEF SUMMARY OF THE DRAWINGS

FIG. 1: Cumulative proportion surviving following treatment with 0.3 and 3 mg/ml 2A10, 3 mg/ml control IgG or PBS. 3 mg/ml 2A10 significantly increases age at death by 16.4 days compared to PBS (95% CI 0.3 to 32.6 days). P<0.05, LSD test post one-way ANOVA.

FIG. 2: Cumulative proportion symptom free following treatment with 0.3 and 3 mg/ml 2A10, 3 mg/ml control IgG or PBS. 0.3 mg/ml 2A10 significantly increases age at onset by 15.5 days compared to PBS (95% CI 2 to 29 days). P<0.05, LSD test post two-way ANOVA.

FIG. 3: MUNE (motor unit number estimation) of the EDL (extensor digitorum longus) muscle in WT and SOD1 mice treated with vehicle or anti-Nogo-A antibody.

FIG. 4: Motor neuron numbers in mouse spinal cord of WT and SOD1 mouse populations treated with vehicle or anti-Nogo-A antibody.

FIG. 5: Maximal tetanic force of the EDL muscle in WT and SOD1 mice treated with vehicle or anti-Nogo-A antibody.

FIG. 6: Maximal twitch (maximum force under a single electrically induced twitch) of the EDL muscle in WT and SOD1 mice treated with vehicle or anti-Nogo-A antibody.

FIG. 7: Weight of the EDL muscle at 90 days in WT and SOD1 mice treated with vehicle or anti-Nogo-A antibody.

FIG. 8: Time taken for the EDL muscle to reach peak force generation following electrical stimulation in WT and SOD1 mice treated with vehicle or anti-Nogo-A antibody.

FIG. 9: Time taken for the EDL muscle to relax after stimulation in WT and SOD1 mice treated with vehicle or anti-Nogo-A antibody.

FIG. 10: Maximum tetanic force of the TA (tibialis anterior) muscle following tetanic stimulation in WT and SOD1 mice treated with vehicle or anti-Nogo-A antibody.

FIG. 11: Maximal twitch of the TA muscle in WT and SOD1 mice treated with vehicle or anti-Nogo-A antibody.

FIG. 12: Weight of the TA muscle at 90 days in WT and SOD1 mice treated with vehicle or anti-Nogo-A antibody.

FIG. 13: Time taken for the TA muscle to reach peak force generation following electrical stimulation in WT and SOD1 mice treated with vehicle or anti-Nogo-A antibody.

FIG. 14: Time taken for the TA muscle to relax after stimulation in WT and SOD1 mice treated with vehicle or anti-Nogo-A antibody.

FIG. 15: MUNE of the EDL muscle in WT and SOD1 mice treated with vehicle (B—PBS), antibody (low dose [LA—3 mg/kg] and high dose [HA—30 mg/kg]), riluzole (R—30 mg/kg), or antibody (low or high dose) plus riluzole (LA+R and HA+R). The treatment groups were the same for each of FIGS. 16 to 20.

FIG. 16: Maximum tetanic force of the TA muscle.

FIG. 17: Maximum twitch in the TA muscle.

FIG. 18: TA muscle weight.

FIG. 19: Time taken for the TA muscle to reach peak force generation following electrical stimulation.

FIG. 20: Time taken for the TA muscle to relax after stimulation.

### DETAILED DESCRIPTION OF THE INVENTION

The cause or trigger of ALS is unknown at present. Sporadic ALS has no known genetic component, however, approximately 20% of fALS cases are caused by dominantly inherited mutations in the protein Cu/Zn superoxide dismutase (SOD1) (Rosen et al. 1993, Nature. 1993; 362:59-62, Andersen 2004, Suppl Clin Neurophysiol. 2004; 57: 211-27). The mutant ‘SOD1’ mouse develops a disease that closely mimics the features of ALS.

Several mouse lines have been generated that overexpress ubiquitously mutant SOD1 (mSOD1) at levels sufficient to induce a motor neuron disease closely resembling human ALS (Gurney et al. 1994, Science 264, 1772-1775). The clinical features observed in these mice are summarized in this summary table taken from Gonzalez de Aguillar et al, 2007, Journal of Neurochemistry, 2007, 101, 1153-1160.

| | | | | Wallerian | Ventral | Motor | | | | Upper | | |
|---|---|---|---|---|---|---|---|---|---|---|---|---|
| | | | Muscle | degeneration | root axon | neuron | Ubiqui- | Astrocyte | Microglial | motor | Pre- | |
| | Muscle | Muscle | fiber type | (sciatic | number | degenera- | tin | prolifer- | prolifer- | neuron | mature | |
| | Weakness | atrophy | switching | nerve) | decrease | tion | staining | ation | ation | signs | death | References |
| Mutant | + | + | + | + | + | + | + | + | + | + | + | (Bruijn et |
| SOD1^{a} | | | | | | | | | | | | al. 2004) |

For these reasons mSOD1 mice (particularly SOD1^{G93A}) may be studied as animal models of ALS.

Two prominent myelin proteins, myelin-associated glycoprotein (MAG) and Nogo-A, have been cloned and identified as inhibitors of neurite outgrowth (Prinjha et al, Nature, 403: 383-384, 2000; GrandPre et al, 2000 Nature, 403:439-444). Nogo-A was originally identified as the antigen for the function blocking antibody IN-1 which had been shown in earlier studies to promote functional recovery in rats following spinal cord injury (Chen et al 2000, Nature, 403(6768):434-9). Subsequent studies from a number of independent laboratories have confirmed the ability of Nogo-A neutralisation in the form of anti-Nogo-A antibodies, active vaccination with Nogo-A derived peptides (Hauben et al 2001, Proc Natl Acad Sci USA, 98(26):15173-8), and Nogo-A gene deletion in mice to enhance functional recovery after spinal cord injury (Kim et al 2003, Neuron, 38(2):187-99; Simonen et al 2003, Neuron, 38(2):201-11).

The present inventors have now shown that pharmacological blockade of Nogo-A (using anti-Nogo-A antibodies) can attenuate signs of disease in SOD1 transgenic mice. This evidence suggests that blockade of Nogo-A could lead to the treatment or prophylaxis of ALS in human patients.

While our own and others' studies have shown that Nogo-A is upregulated in the spinal cord and in the affected muscles of SOD1 transgenic mice and ALS patients, the functional significance of this has remained unclear and a matter of significant controversy. Jokic et at (EMBO Reports, 2006:7(11), 1162-1167) have subsequently shown that a genetic cross between SOD1 transgenic mice and Nogo-A deficient mice caused a small but significant delay in disease onset, improvement in mouse survival and increase in motor neuron numbers. However, a number of important questions remained unanswered, including whether these benefits were a function of the loss of Nogo-A during development or indeed a function of any compensatory changes in Nogo-B and Nogo-C, which are known to be upregulated in these mice and also known to change in ALS (Simonen et al (2003) Neuron 38 201-211; DuPuis et al (2002) Neurobiol Dis 10 359-365).

The present inventors have now unexpectedly found that treatment of SOD1 transgenic mice with an anti-Nogo-A antibody can result in significantly delayed disease onset, time to death, improved muscle physiology and motor neuron survival. Furthermore despite their very different modes of action the inventors have unexpectedly found that in a number of measures of muscle function there is evidence for an additive and even synergistic effect of anti-Nogo-A and the anti-glutamatergic compound riluzole.

Three forms of human NOGO have been identified: NOGO-A having 1192 amino acid residues (GenBank accession no. AJ251383, SEQ ID No. 10); NOGO-B, a splice variant which lacks residues 186 to 1004 in the putative extracellular domain (GenBank accession no. AJ251384) and a shorter splice variant, NOGO-C, which also lacks residues 186 to 1004 and also has smaller, alternative amino terminal domain (GenBank accession no. AJ251385) (Prinjha et al (2000) supra). Nogo-A is a potent inhibitor of neurite outgrowth.

A “Nogo-A antagonist” as used herein refers to any compound that inhibits, blocks, attenuates, or interferes with any pathway elicited, either directly or indirectly, by Nogo-A. Thus, the term “antagonists” is intended to include, but is not limited to, molecules which neutralise the effect of Nogo-A.

“Nogo-A antibody” as used here in refers to any antibody or variant form thereof, including but not limited to, antibody fragment, domain antibody or single chain antibody capable of binding to Nogo-A. A Nogo-A antagonist may be an antibody antagonist such as a neutralising anti-Nogo-A antibody. A Nogo-A antibody may be murine, chimeric, humanized, or fully human antibody or fragment thereof.

“Antibody Antagonists” as used herein refers to any antibody or variant form thereof, including but not limited to, antibody fragment, domain antibody or single chain antibody capable of reducing the activity of a given pathway, enzyme, receptor or ligand, such as a Nogo-A pathway. Antibody antagonists include antibodies in a conventional immunoglobulin format (IgA, IgD, IgE, IgG, IgM), and also fragments thereof or any other “antibody-like” format that binds to human Nogo-A (for example, single chain Fv, Fc, Fd, Fab, F(ab)₂, diabodies, Tandabs™, domain antibodies (dAbs), etc. (for a summary of alternative “antibody” formats see Holliger and Hudson, Nature Biotechnology, 2005, Vol 23, No. 9, 1126-1136)). The terms Fv, Fc, Fd, Fab, or F(ab)₂ are used with their standard meanings (see, e.g., Harlow et al., Antibodies A Laboratory Manual, Cold Spring Harbor Laboratory, (1988)).

“Neutralising” and grammatical variations thereof refers to inhibition, either total or partial, of any NOGO function.

“NOGO-function” as used herein refers to any biological activity elicited by a Nogo protein including, but not limited to, triggering any NOGO-pathway, binding to neurones and inhibition of neurite growth.

“Treatment” as used herein refers to the reduction or elimination of disease symptoms associated with and/or causes of amyotrophic lateral sclerosis, including the reduction in or elimination of the progressive degeneration of the neurons in the corticospinal tracts, the denervation of muscle fibres, and/or muscle weakness and/or spasticity.

“Prophylaxis” as used herein refers to the retardation, prevention or minimization of disease symptoms associated with amyotrophic lateral sclerosis, including the retardation, prevention or minimization of the progressive degeneration of the neurons in the corticospinal tracts, the denervation of muscle fibres, and/or muscle weakness and/or spasticity.

“Anti-glutamate activity” refers to an ability of a compound to inhibit partially or fully any biological activity elicited by a glutamate receptor, including reducing the biological activity of glutamate receptors. Compounds with anti-glutamate activity are also known as anti-glutamatergic compounds. A compound with anti-glutamate activity may therefore be, inter alia, a glutamate receptor antagonist or an antagonist of glutamate release from presynaptic terminals.

Glutamate is the main excitatory neurotransmitter in the CNS. An excess of glutamate over-stimulates the glutamate receptors, which can lead to neuronal degeneration. This cellular mechanism is known as excitotoxicity (Leigh et al., Neurology (1996) 47:S221-S227), and is believed to be due primarily to increased Ca²⁺ permeability and delayed desensitization of the glutamate receptors. Abnormal glutamate release has been implicated in a number of neuropathological conditions and widespread alterations in glutamate levels have been observed in the CNS of ALS patients.

Glutamate receptors are categorized into ionotropic and metabotropic glutamate receptors, based on their structure, function and pharmacology. The ionotropic glutamate receptors, which are ion channels allowing cation flow into the neurons, are subdivided into the N-methyl-D-aspartic acid (NMDA) subtype, the alpha-amino-3-hydroxy-5-methylisoxazole-4-propionic acid (AMPA) subtype, the kainic acid (KA) subtype and the delta subtype (the delta2 glutamate-like receptor undergoes similar conformational changes as other ionotropic glutamate receptors, MacLean, J Neurosci. 2009 29(21):6767-8). The population of glutamate receptors in motor neurones is distinct from other cell types; in most neurones, the NMDA subtype predominantly mediates glutamate cytotoxicity; in motor neurones, the AMPA/kainite subclass is potentially more important.

Riluzole (Rilutek®, 2-Amino-6-(trifluoromethoxy)benzothiazole; 6-Trifluoromethoxy-2-aminobenzothiazole; 6-(Trifluoromethoxy)-1,3-benzothiazol-2-amine, CAS Registry Number 1744-22-5), inhibits glutamate release from presynaptic terminals, and has demonstrated neuroprotective effects against excitotoxic damage in animal models of brain damage (Wahl et al. Eur. J. Pharmacol. (1993), 230:209-214). Although the precise mechanism of Riluzole is unknown, it is believed to have multiple effects on the ionotropic glutamate receptor system, including: inhibiting the G-protein-dependent release of glutamate to the synaptic cleft (Kwon et al, Anesth Analg (1998) 86:128-133); reducing the release of glycine, resulting in the reduction in N-methyl-d-aspartate (NMDA) channel activity (Umemiya and Berger, Br J Pharmacol (1995) 116:3227-3230); diminishing the sensitivity of postsynaptic AMPA receptors (Centonze et al, Neuropharmacology (1998) 37:1063-1070); prolonging the inactivation state of the α-subunit of the Na⁺ (Herbert et al, Mol Pharmacol (1994) 45:1055-1060 and Stutzmann at al. Eur J Pharmacol (1991) 193:223-229), attenuating the NMDA-mediated excitation (Kretschmer et al. Naunyn Schmiedebergs Arch Pharmacol (1998) 358:181-190); preventing Ca²⁺ mobilization by activated G proteins (Kretschmer et al. supra), and blocking indirectly postsynaptic responses of glutamatergic receptors (Yoshida et al., Epilepsy Res (2001) 46:101-109).

Talampanel ([(R)-7-acetyl-5-(4-aminophenyl)-8,9-dihydro-8-methyl-7H-1,3-dioxolo[4,5-h][2,3]benzodiazepine], CAS Registry Number 161832-65-1) is a negative allosteric modulator of AMPA receptors. The 2,3-benzodiazepines have been shown to be neuroprotective in neuronal cultures exposed to kainite or AMPA (Szénási and Hársing Jr., Drug Discovery Today (2004) 69-76).

Additional anti-glutamatergic compounds include but are not limited to: TRO19622 (Cholest-4-en-3-one, oxime); ONO-2506 (Cereact™, Arundic acid, (R)-(−)-2-propyloctanoic acid); memantine (Namenda™, 1-amino-3,5-dimethyl-adamantane), ceftriaxone (5-Thia-1-azabicyclo[4.2.0]oct-2-ene-2-carboxylic acid, 7-[[(2-amino-4-thiazolyl)(methoxyimino)acetyl]amino]-8-oxo-3-[[(1,2,5,6-tetrahydro-2-methyl-5-,6-dioxo-1,2,4-triazin-3-yl)thio]methyl]-, disodium salt, [6R-[6 a,7b(Z)]]-, hydrate, (2:7)), NBQX (1,2,3,4-tetrahydro-6-nitro-2,3-dioxo-benzo[f]quinoxaline-7-sulfonamide) and NAALADase inhibitors such as GPI-16062 (Guilford Pharmaceuticals).

“Refractory” to treatment with a compound having anti-glutamate activity, such as riluzole, refers to an inadequate or unsustained response to previous or current treatment with said compound. For instance, a subject that is refractory to treatment with riluzole includes, therefore, a subject that previously responded to such treatment, but no longer responds to said treatment to the same degree. A refractory subject includes a subject whose illness regresses back to its former state, with the return of disease symptoms following an apparent recovery or partial recovery.

Patients with an inadequate response to riluzole therapy typically have severe and/or longer standing disease. An “inadequate response” may be due to inadequate efficacy of the treatment. An inadequate response to a specific treatment may be established by studying one or more clinical markers, which are associated with the disease or disorder, known to those skilled in the art. Accordingly, an inadequate response can be determined by a clinician skilled in treating ALS.

As used herein “co-administration” or “co-administering” refers to administration of two or more compounds to the same patient. Co-administration of such compounds may be simultaneous or at about the same time (e.g., within the same hour) or it may be within several hours or days of one another. For example, a first compound may be administered once weekly while a second compound is co-administered daily. Typically there will be a time period during which both the first and second compounds (or all of the co-administered compounds) simultaneously exert their biological effects.

Monoclonal antibodies which bind to NOGO are described in inter alia WO04/052932, WO2005/028508, WO2005/061544 and WO2007/068750, the contents of which are incorporated herein in their entirety. WO2005/061544 discloses the murine anti-Nogo-A monoclonal antibodies 2A10, 15C3 and 2C4, and provides data showing the ability of these antibodies to block the neurite-outgrowth inhibitory activity of NOGO-A56. WO2007/068750 discloses humanised antibodies which bind to human NOGO with high affinity, including H28L16, H28L13 and H27L16, and provides data showing that these humanised antibodies have an activity comparable to parent antibody 2A10 in the neurite-outgrowth assay.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl. Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

The term “donor antibody” refers to a non-human antibody which contributes the amino acid sequences of its variable regions, CDRs, or other functional fragments or analogues thereof to the humanised antibody, and thereby provide the humanised antibody with the antigenic specificity and neutralizing activity characteristic of the donor antibody.

The term “acceptor antibody” refers to an antibody heterologous to the donor antibody, which provides the amino acid sequences of its heavy and/or light chain framework regions and/or its heavy and/or light chain constant regions to the humanised antibody. The acceptor antibody may be derived from any mammal provided that it is non-immunogenic in humans. Preferably the acceptor antibody is a human antibody.

Alternatively, humanisation maybe achieved by a process of “veneering”. A statistical analysis of unique human and murine immunoglobulin heavy and light chain variable regions revealed that the precise patterns of exposed residues are different in human and murine antibodies, and most individual surface positions have a strong preference for a small number of different residues (see Padlan E. A. et al; (1991) Mol. Immunol. 28, 489-498 and Pedersen J. T. et al (1994) J. Mol. Biol. 235; 959-973). Therefore it is possible to reduce the immunogenicity of a non-human Fv by replacing exposed residues in its framework regions that differ from those usually found in human antibodies. Because protein antigenicity can be correlated with surface accessibility, replacement of the surface residues may be sufficient to render the mouse variable region “invisible” to the human immune system (see also Mark G. E. et at (1994) in Handbook of Experimental Pharmacology vol. 113: The pharmacology of monoclonal Antibodies, Springer-Verlag, pp 105-134). This procedure of humanisation is referred to as “veneering” because only the surface of the antibody is altered, the supporting residues remain undisturbed. A further alternative approach is set out in WO04/006955.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p877-883.

Anti-Nogo-A antibodies particularly useful in the method according to the present invention include H28L16 (SEQ ID NO:2 and SEQ ID NO:4), H28L13 (SEQ ID NO:2 and SEQ ID NO:3) and H27L16 (SEQ ID NO:1 and SEQ ID NO:4). The full length (FL) IgG1 heavy chain sequences H27 and H28 are shown as SEQ ID NOs 1 and 2, respectively, below.

SEQ ID NO: 1:
Heavy chain humanised construct H27
MGWSCIILFLVATATGVHSQVQLVQSGAEVKKPGASVKVSCKASGYTF
TSYWMHWVKQRPGQGLEWIGNINPSNGGTNYNEKFKSKATLTVDKSTS
TAYMELSSLRSEDTAVYYCELMQGYWGQGTLVTVSSASTKGPSVFPLA
PSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHT
CPPCPAPELAGAPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLV
KGFYPSDIAVEWESNGQPENNYKTTPPVLDSDGSFFLYSKLTVDKSRW
QQGNVFSCSVMHELAHNHYTQKSLSLSPGK
SEQ ID NO: 2:
Heavy chain humanised construct H28
MGWSCIILFLVATATGVHSQVQLVQSGAEVKKPGASVKVSCKASGYTF
TSYWMHWVRQAPGQGLEWIGNINPSNGGTNYNEKFKSKATMTRDTSTS
TAYMELSSLRSEDTAVYYCELMQGYWGQGTLVTVSSASTKGPSVFPLA
PSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHT
CPPCPAPELAGAPSVFLFPPKPKDTLMISRTPEVTCVVVDVSHEDPEV
KFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLV
KGFYPSDIAVEWESNGQPENNYKTTPPVLDSDGSFFLYSKLTVDKSRW
QQGNVFSCSVMHEALHNHYTQKSLSLSPGK

The FL IgG1 light chain sequences L13 and L16, are shown as SEQ ID NOs 3 and 4, respectively, below.

SEQ ID NO: 3:
Light chain construct L13
MGWSCIILFLVATATGVHSDIVMTQSPLSLPVTLGQPASISCRSSKSL
LYKDGKTYLNWFQQRPGQSPQLLIYLMSTRASGVPDRFSGGGSGTDFT
LKISRVEAGDVGVYYCQQLVEYPLTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 4:
Light chain construct L16
MGWSCIILFLVATATGVHSDIVMTQSPLSNPVTLGQPVSISCRSSKSL
LYKDGKTYLNWFLQRPGQSPQLLIYLMSTRASGVPDRFSGGGSGTDFT
LKISRVEAEDVGVYYCQQLVEYPLTFGQGTKLEIKRTVAAPSVFIFPP
SDEQLKSGTASVVCLLNNFYPREAKVQWKVDNALQSGNSQESVTEQDS
KDSTYSLSSTLTLSKADYEKHKVYACEVTHQGLSSPVTKSFNRGEC

In another embodiment, the Nogo-A antagonist is an antibody, or fragment thereof, which is capable of binding to human Nogo-A protein, or a fragment thereof, such as GST-NOGO-A56 protein (SEQ ID NO. 5), in an ELISA assay, wherein the binding of the antibody, or fragment thereof, to the human NOGO protein, or fragment thereof, in the ELISA assay is reduced in the presence of a peptide having the following sequence VLPDIVMEAPLN (SEQ ID NO. 6) (human Nogo 610-621aa), or TPSPVLPDIVMEAPLN (SEQ ID NO. 7) or VLPDIVMEAPLNSAVP (SEQ ID NO. 8), and is not reduced in the presence of an irrelevant peptide, for instance a peptide from human Nogo that does not overlap with SEQ ID NO. 6 (such as SEQ ID NO. 9, YESIKHEPENPPPYEE).

SEQ IN NO: 5:
Amino acids 586-785 of human NOGO A (NOGO-A56)
fused to GST
MSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLE
GAVLDIRYGVSRIAYSKDFETLKVDFLSKLPEMLKMFEDRLCHKTYLN
GDHVTHPDFMLYDALDVVLYMDPMCLDAFPKLVCFKKRIEAIPQIDKY
LKSSKYIAWPLQGWQATFGGGDHPPKSDLEVLFQGPLGSMQESLYPAA
QLCPSFEESEATPSPVLPDIVMEAPLNSAVPSAGASVIQPSSSPLEAS
SVNYESIKHEPENPPPYEEAMSVSLKKVSGIKEEIKEPENINAALQET
EAPYISIACDLIKETKLSAEPAPDFSDYSEMAKVEQPVPDHSELVEDS
SPDSEPVDLFSDDSIPDVPQKQDETVMLVKESLTETSFESMIEYENKE
LERPHRD
SEQ ID NO. 6:
VLPDIVMEAPLN
SEQ ID NO. 7:
TPSPVLPDIVMEAPLN
SEQ ID NO. 8:
VLPDIVMEAPLNSAVP
SEQ ID NO. 9:
YESIKHEPENPPPYEE
SEQ ID NO. 10:
Human Nogo-A
MEDLDQSPLVSSSDSPPRPQPAFKYQFVREPEDEEEEEEEEEEDEDED
LEELEVLERKPAAGLSAAPVPTAPAAGAPLMDFGNDFVPPAPRGPLPA
APPVAPERQPSWDPSPVSSTVPAPSPLSAAAVSPSKLPEDDEPPARPP
PPPPASVSPQAEPVWTPPAPAPAAPPSTPAAPKRRGSSGSVDETLFAL
PAASEPVIRSSAENMDLKEQPGNTISAGQEDFPSVLLETAASLPSLSP
LSAASFKEHEYLGNLSTVLPTEGTLQENVSEASKEVSEKAKTLLIDRD
LTEFSELEYSEMGSSFSVSPKAESAVIVANPREEIIVKNKDEEEKLVS
NNILHNQQELPTALTKLVKEDEVVSSEKAKDSFNEKRVAVEAPMREEY
ADFKPFERVWEVKDSKEDSDMLAAGGKIESNLESKVDKKCFADSLEQT
NHEKDSESSNDDTSFPSTPEGIKDRSGAYITCAPFNPAATESIATNIF
PLLGDPTSENKTDEKKIEEKKAQIVTEKNTSTKTSNPFLVAAQDSETD
YVTTDNLTKVTEEVVANMPEGLTPDLVQEACESELNEVTGTKIAYETK
MDLVQTSEVMQESLYPAAQLCPSFEESEATPSPVLPDIVMEAPLNSAV
PSAGASVIQPSSSPLEASSVNYESIKHEPENPPPYEEAMSVSLKKVSG
IKEEIKEPENINAALQETEAPYISIACDLIKETKLSAEPAPDFSDYSE
MAKVEQPVPDHSELVEDSSPDSEPVDLFSDDSIPDVPQKQDETVMLVK
ESLTETSFESMIEYENKEKLSALPPEGGKPYLESFKLSLDNTKDTLLP
DEVSTLSKKEKIPLQMEELSTAVYSNDDLFISKEAQIRETETFSDSSP
IEIIDEFPTLISSKTDSFSKLAREYTDLEVSHKSEIANAPDGAGSLPC
TELPHDLSLKNIQPKVEEKISFSDDFSKNGSATSKVLLLPPDVSALAT
QAEIESIVKPKVLVKEAEKKLPSDTEKEDRSPSAIFSAELSKTSVVDL
LYWRDIKKTGVVFGASLFLLLSLTVFSIVSVTAYIALALLSVTISFRI
YKGVIQAIQKSDEGHPFRAYLESEVAISEELVQKYSNSALGHVNCTIK
ELRRLFLVDDLVDSLKFAVLMWVFTYVGALFNGLTLLILALISLFSVP
VIYERHQAQIDHYLGLANKNVKDAMAKIQAKIPGLKRKAE
SEQ ID 11:
2A10 VH humanised construct H20
QVQLVQSGAEVKKPGASVKVSCKASGYTFTSYWMHWVRQAPGQGLEWI
GNINPSNGGTNYNEKFKSKATMTRDTSTSTAYMELSSLRSEDTAVYYC
ELGQGYWGQGTLVTVSS
SEQ ID 12:
VH humanised construct H27
QVQLVQSGAEVKKPGASVKVSCKASGYTFTSYWMHWVKQRPGQGLEWI
GNINPSNGGTNYNEKFKSKATLTVDKSTSTAYMELSSLRSEDTAVYYC
ELMQGYWGQGTLVTVSS
SEQ ID 13:
VH humanised construct H28
QVQLVQSGAEVKKPGASVKVSCKASGYTFTSYWMHWVRQAPGQGLEWI
GNINPSNGGTNYNEKFKSKATMTRDTSTSTAYMELSSLRSEDTAVYYC
ELMQGYWGQGTLVTVSS
SEQ ID 14:
2A10 VL humanised construct L13
DIVMTQSPLSLPVTLGQPASISCRSSKSLLYKDGKTYLNWFQQRPGQS
PQLLIYLMSTRASGVPDRFSGGGSGTDFTLKISRVEAEDVGVYYCQQL
VEYPLTFGQGTKLEIK
SEQ ID 15:
2A10 VL humanised construct L16
DIVMTQSPLSNPVTLGQPVSISCRSSKSLLYKDGKTYLNWFLQRPGQS
PQLLIYLMSTRASGVPDRFSGGGSGTDFTLKISRVEAEDVGVYYCQQL
VEYPLTFGQGTKLEIK
SEQ ID NO: 16:
Variable part of heavy chain of 11C7 with leader
sequence
MDFGLIFFIVGLLKGVQCEVKLLESGGLVQPGGSLKLSCVVSGFDFRR
NWMSWVRQAPGKGLEWIGEINPDSSKINYTPSLKDKFIISRDNAKNTL
YLQVSTVRSEDTALYTCVRPVWMYAMDYWGQGTSVTVSSAKTTPPSVY
PLAPGSAAQTNSMVTLGCLVKGYFPEPVTVTWNSGSLSSGVHTFPAVL
QSDLYTLSSSVTVPS STWPSETVTCNVA
SEQ ID NO: 17:
Light chain of 11C7 with leader sequence
MSPAQFLFLLVLWIRETSGDVLLTQTPLTLSITIGQPASISCKSSQSL
LHSDGKTYLNWLLQRPGQSPKRLIYLVSKLDSGVPDEFTGSGSGTDFT
LKISRVEAGDLGLYYCWQGTHFPQTFGGGTKLEIKRADAAPTVSIFPP
SSGQLTSGGASVVCFLNNFYPKDINVKWKIDGSERQNGVLNSWDQDSK
DSTYSMSSTLTLTKD EYERHNSYTCEATHKTSTSPIVKSFNRGEC
SEQ ID NO: 18:
Variable part of heavy chain of 6A3 with leader
sequence
MEFGLSWVFLVAILEGVQCEVQLVESGGGLVQPGGSLRLSCAASGFTF
SNYWMSWVRQAPGKGLEWVATIKQDGSQKNYVDSVKGRFTISRDNAKN
SLYLRLNSLRAEDTAVYYCATELFDLWGRGSLVTVSSASTKGPSVFPL
APSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTH
TCPPCP
SEQ ID NO: 19:
Variable part of light chain of 6A3 with leader
sequence
MEAPAQLLFLLLLWLPDTTGEIVLTQSPATLSLSPGERATLSCRASQS
VSSYLAWYQQKPGQAPRLLIYDASNRATGIPARFSGSGSGTDFTLTIS
SLEPEDFAVYYCQQRSNWPITFGQGTRLEIKRTVAAPSVFIFPPSDEQ
LKSGTASVVCLLNNFYPREAKVQWKVDNALQSGNSQESVTEQDSKDST
YSLSSTLTLSKADYEKHKVYACEVTHQGLSSPVTKSFNRGEC

The following examples illustrate but do not limit the invention.

### Example 1

### In Vivo ALS Model Survival and Onset Study with 2A10 and 2C4

The ability of 2A10 to modify disease progression was investigated in a mouse model of ALS (reviewed in Benatar 2007, Neurobiol Dis. 26(1):1-13).

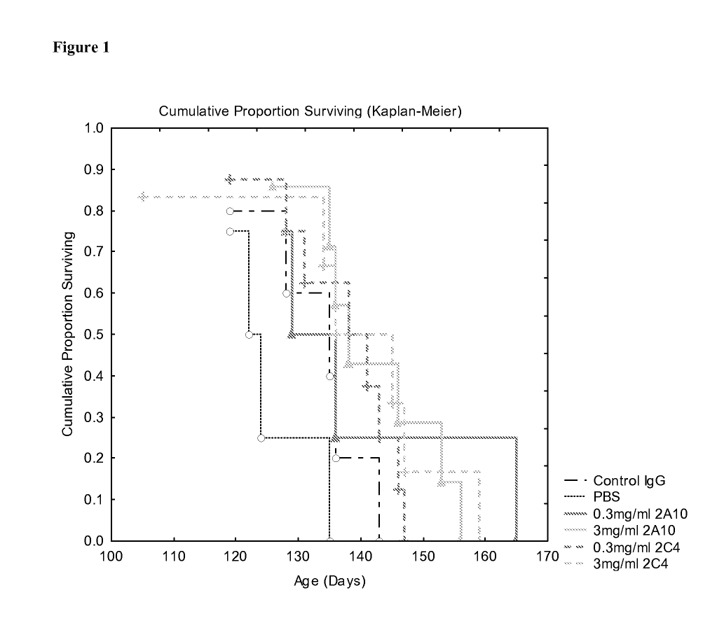

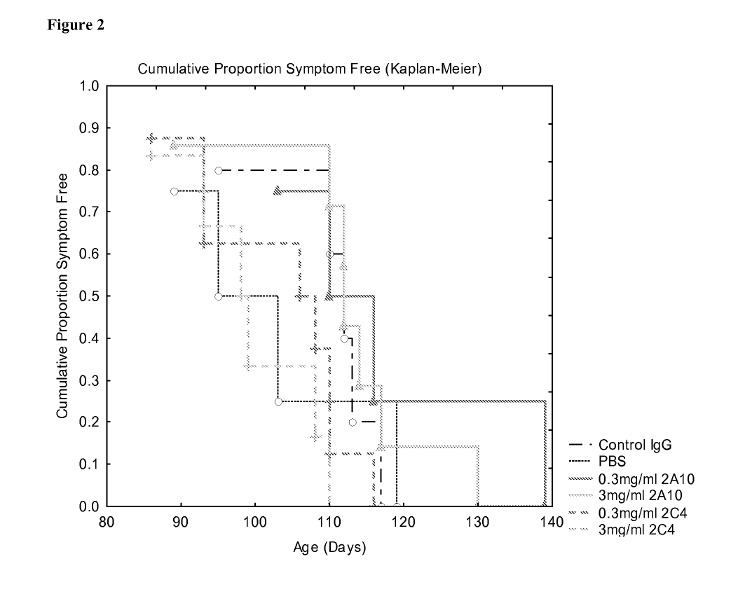

FIG. 1 and FIG. 2 respectively show the cumulative proportion of mice surviving and cumulative proportion symptom free following treatment with 2A10 (0.3 and 3.0 mg/ml, equivalent to 3.0 and 30 mg/kg respectively) in comparison to PBS and a Control IgG (3.0 mg/ml). The results of this study show that 3 mg/ml 2A10 significantly increases age at death by 16.4 days compared to PBS (95% CI 0.3 to 32.6 days, P<0.05) and that 0.3 mg/ml 2A10 significantly increases age at onset by 15.5 days compared to PBS (95% CI 2 to 29 days, P<0.05). These results were confirmed using another anti-NOGO-A monoclonal antibody 2C4 (disclosed in WO2005/061544) which binds to a distinct epitope.

The anti-Nogo-A antibody 2A10 therefore prolongs survival in mice in an ALS model. This result suggests that Nogo-A blockade, particularly with 2A10, and humanised variants of 2A10, such as H28L16 (SEQ ID NO:2 and SEQ ID NO:4), H28L13 (SEQ ID NO:2 and SEQ ID NO:3) and H27L16 (SEQ ID NO:1 and SEQ ID NO:4), which share the same epitope of 2A10 (and also other anti-Nogo-A antibodies which share the same epitope as 2A10), would be useful in the treatment or prophylaxis of ALS in humans, particularly when the Nogo-A blockade therapy is combined with riluzole therapy.

### Example 2

### In Vivo ALS Model Muscle Physiology Studies with 2A10

An additional study was performed in SOD1 mice comparing 30 mg/kg anti-Nogo-A 2A10 with vehicle treated SOD1 and wild-type mice at day 90, and produced a package of data that was consistent with a significant treatment related benefit in a number of important and clinically relevant measures of mouse muscle physiology.

### Materials and Methods

Transgenic mice overexpressing human Cu/Zn-SOD G93A mutations ((B6SJL-TgN (SOD1-G93A) 1 Gur) originally purchased from Jackson Laboratories (Ben Harbor, Me., USA), were bred and maintained in Biological Services, UCL ION. SOD1^{G93A} hemizygous males are crossed with wildtype F1 (SJL×C57BL/6) females, as recommended by the Jackson Laboratory (hemizygous SOD1^{G93A} females are infertile). In our colony, male SOD1^{G93A} mice have an average lifespan of 123 days and female SOD1^{G93A} mice have an average lifespan of 130 days. In this study, only female animals were examined. Transgenic SOD1^{G93A} mice were genotyped by amplification of mouse ear or tail DNA by polymerase chain reaction at weaning age. For each animal the genotype was confirmed at the end of the study, at around 3 months of age.

All experiments were carried out under the guidance issued by the Medical Research Council in Responsibility in the Use of Animals for Medical Research (1993) and under licence from the UK Home Office, following ethical review by UCL ION.

### Treatment Protocol: Anti-Nogo A Treatment in SOD1^{G93A} Mice

Animals were divided into 4 experimental groups consisting of SOD1 and wildtype (WT) littermates. Two groups of WT littermates (n=10) served as controls and were treated with vehicle (PBS: Treatment B) or Anti-Nogo-A antibody at 30 mg/kg (Treatment HA). Two groups of SOD1 mice (n=10) were treated with vehicle (PBS: Treatment B) or Anti-Nogo-A antibody at 30 mg/kg (Treatment HA). Thus, the following experimental groups were established:
Group I: WT treated with vehicle (PBS) (n=10) (Treatment B)
Group II: WT treated with Anti-Nogo Antibody (30 mg/kg) (n=10) (Treatment HA)
Group III: SOD1 treated with vehicle (PBS) (n=10) (Treatment B)
Group IV: SOD1 treated with Anti-Nogo Antibody (30 mg/kg) (Treatment HA)

Anti-Nogo A antibody or vehicle (PBS) was administered by i.p. injections weekly, starting from 70 days of age until 90 days of age (3 injections).

### Assessment of Disease Progression

### 1. Behaviour and Body Weight Assessment:

Mice were observed daily and weighed twice weekly. Motor performance was assessed from 70 days of age using grip strength testing. The grip strength test assessed neuromuscular function by measuring, with an electronic digital force gauge, the peak amount of force an animal applied in grasping a 10 cm×8 cm wire grid attached to a pull bar (Bioseb Instruments). The mouse was placed on the flat wire grid connected to the force gauge and held on with front and hind paws. It was held by the base of the tail and was gently pulled away from the grid until the mouse released its grip at which point peak tension on the pull bar was recorded. The mean of 4 measurements was determined for each mouse on each day of testing. Further details of the Standard Operating Procedure for grip strength that we followed can be found at the Eumorphia website: http://www.eumorphia.org/EMPReSS/servlet/EMPReSS.Frameset

### 2. In Vivo Assessment of Muscle Force and Motor Unit Number

### i) Maximum Force and Motor Unit Survival

The maximum force of the tibialis anterior (TA) and extensor digitorium longus (EDL) muscles of each animal was assessed at 90 days of age. The animals were anesthetized (4.5% chloral hydrate solution, 1 ml/100 g body weight, i.p.; Sigma-Aldrich, Poole, UK) and prepared for isometric tension recordings of muscle contraction (Kieran and Greensmith, 2004). The distal tendons of hind-limb TA and EDL muscles were exposed, dissected free from surrounding tissue, and cut. The sciatic nerve was exposed and sectioned, and all of its branches were cut apart from the deep peroneal nerve, which innervates the TA and EDL muscles. The hind limbs of the animals were rigidly secured to the table with stainless steel pins, and the distal tendons of the TA and EDL muscles were attached to an isometric force transducer (Dynamometer UFI Devices, Welwyn Garden City, UK) via thread. Once attached, the length of each muscle was adjusted to obtain maximal twitch tension. Both muscles and nerves were kept moist with saline, and experiments performed at room temperature. Isometric contractions were elicited by stimulating the nerve to TA and EDL using square-wave pulses of 0.02-ms duration and supramaximal intensity via platinum electrodes. Contractions were elicited by trains of stimuli at a frequency of 20, 40, and 80 Hz. Twitch, maximum tetanic tension, time to peak, and half-relaxation time values were measured.

The number of motor units in both EDL muscles was assessed by applying stimuli of increasing intensity to the motor nerve, resulting in stepwise increments in twitch tension, due to successive recruitment of motor axons.

### ii) Fatigue Test

At the end of the isometric tension recordings, the resistance of the EDL muscles to fatigue during repeated stimulation was tested. The EDL muscles were stimulated at 40 Hz for 250 ms every second and the contractions were recorded on a pen recorder (Multitrace 2; Lectromed). The decrease in tension after 3 min of stimulation was measured and the fatigue index (F.I.) was calculated as (initial tetanic tension−tetanic tension after stimulation)/initial tetanic tension). A F.I. approaching 1 indicates that the muscle is very fatiguable.

### 3. Muscle Histochemistry

At the end of each experiment, the TA and EDL muscles were removed, weighed, and snap frozen in isopentane cooled in liquid nitrogen and stored at −80° C. until processing. Serial cross sections (10 μm) of TA muscle were cut on a cryostat and stained for succinate dehydrogenase (SDH) activity to determine the oxidative capacity of the muscle fibres, as described previously (Kieran and Greensmith, 2004).

### 4. Motoneuron Survival

Following transcardial perfusion with 4% paraformaldehyde (4% PFA), the lumbar region of the spinal cord was removed, post-fixed in 4% PFA for 6 hours and submerged in 30% sucrose for a minimum of 8 hours. Serial cross sections (20 μm) were cut using a cryostat and stained with gallocyanin, a Nissl stain. The number of Nissl-stained motoneurons in the sciatic motor pool of every third section (n=60) between the L2 and L5 levels of the spinal cord were counted. Only large, polygonal neurons with a distinguishable nucleus and nucleolus and clearly identifiable Nissl structure were included in the counts.

### 5. Microscopy

Spinal cord and muscle sections were examined under a light microscope (Leica DMR) using Leica HC PL Fluotar objectives (10×, 20× and 40× magnification). Images were captured using a Nikon E995 digital camera and the images downloaded into Adobe Photoshop CS. To optimise image contrast, Levels Adjustment operations were performed, but no other image manipulations were made.

### 6. Statistics

Statistical significance among the groups was assessed using a Mann-Whitney U test. Significance was set at P<0.05

### Results

i. EDL Motor Unit Number Estimation

Electrical stimulation of Extensor digitorum longus (EDL) muscle with increasing intensity is able to induce activation of successively greater motor units with each producing a characteristic trace. Summation of the traces can be used to produce an estimate of surviving motor unit numbers. Disease progression in SOD1 mice results in a significant and progressive reduction in motor unit traces. Treatment with 30 mg/kg anti-Nogo-A 2A10 resulted in a significant improvement in motor unit numbers (p value 0.0494). The results are shown in FIG. 3.

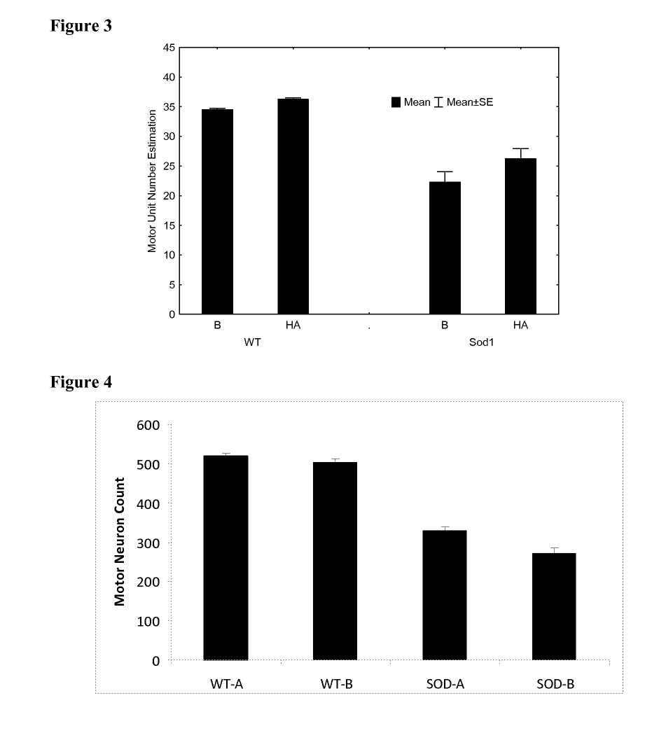

It was highly encouraging but unexpected that this improvement in motor unit numbers seen in the electrical stimulation assay correlated perfectly with an equivalent improvement in motor neuron numbers in the spinal cord shown in FIG. 4 (SOD A vs SOD B p value 0.003). This direct evidence of a CNS neuroprotective activity in the spinal cord following systemic administration of an anti-Nogo-A antibody in a disease model provides strong rationale for the potential to see similar beneficial activity in ALS patients. It also suggests that clinical measures such as MUNE may be useful in the early detection of neuroprotective benefits.

This package of data is consistent with the use of anti-Nogo-A antibodies in the treatment of ALS and other muscle diseases in which Nogo-A has been shown to be upregulated in muscle biopsies, such as those described supra. The ability of systemic anti-Nogo-A treatment to result in significant neuroprotection in the CNS is further consistent with its therapeutic use in a wide range of neurological diseases, such as those described supra.

ii. EDL Maximum Tetanic Force

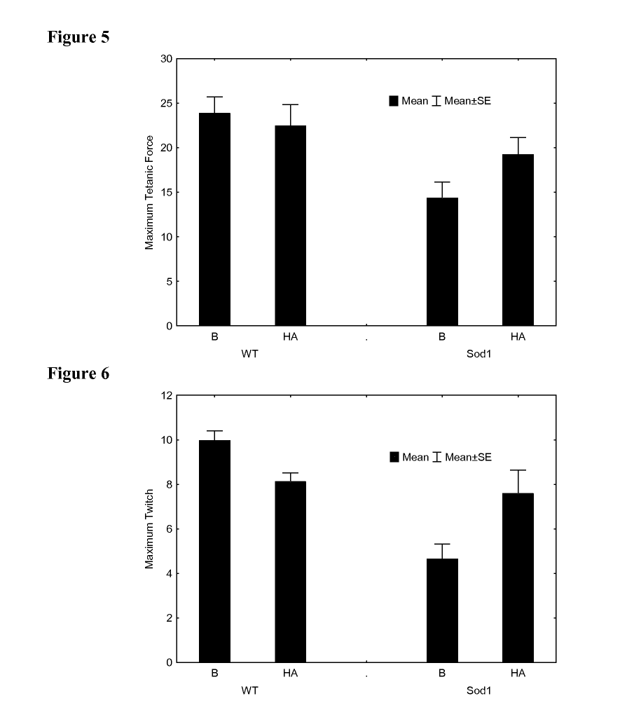

The maximum tetanic force generated by the EDL was partially improved by anti-Nogo-A treatment (FIG. 5).

iii. EDL Maximum Twitch

The maximum force generated during a single electrically induced twitch was measured and found to be significantly improved in the anti-Nogo-A treated group (p value 0.01). The results are shown in FIG. 6.

iv. EDL Muscle Weight

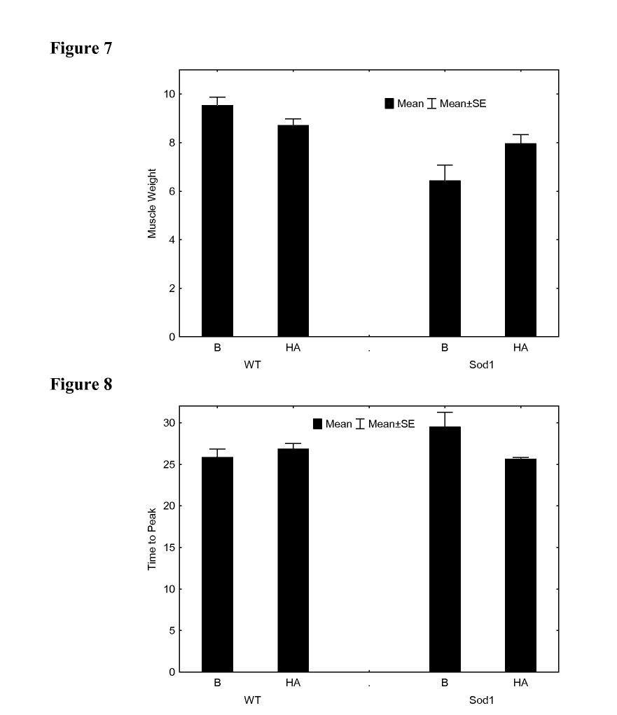

While there is not a large effect on muscle weight loss at 90 days in SOD1 mice there was a significant improvement in EDL muscle weight in anti-Nogo-A treated mice in this group (p value 0.0276). The results are shown in FIG. 7.

v. EDL Time to Peak

The time taken to reach the peak force generation following electrical stimulation in the SOD1 mice at 90 days shows a small but significant delay at 90 days that was reversed by anti-Nogo-A treatment (p value 0.0232). The results are shown in FIG. 8.

vi. EDL Time to ½ Relaxation

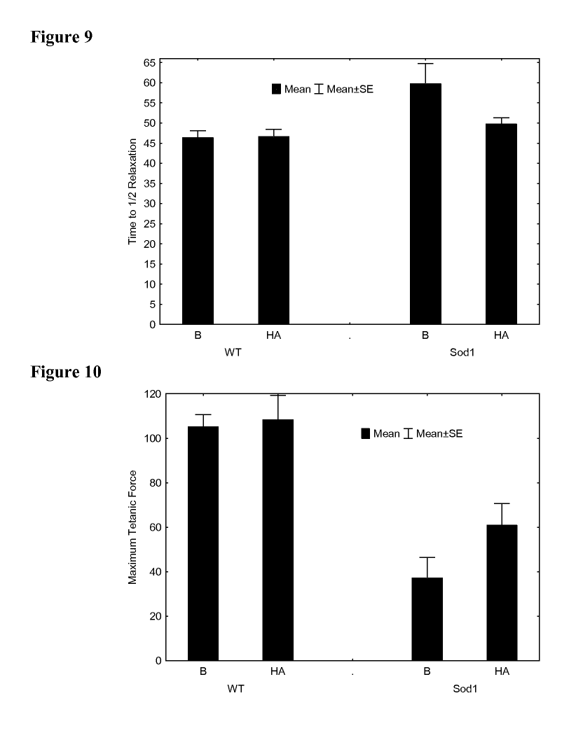

The time taken for the EDL to relax after stimulation is increased in SOD1 mice and this was significantly reduced by anti-Nogo-A treatment (p value 0.0312). The results are shown in FIG. 9.

vii. TA Maximum Tetanic Force

The maximum force generated by the TA muscle following tetanic stimulation is reduced in SOD1 mice, showing a treatment-related trend towards increased maximum tetanic force in the HA group (FIG. 10).

viii. TA Maximum Twitch

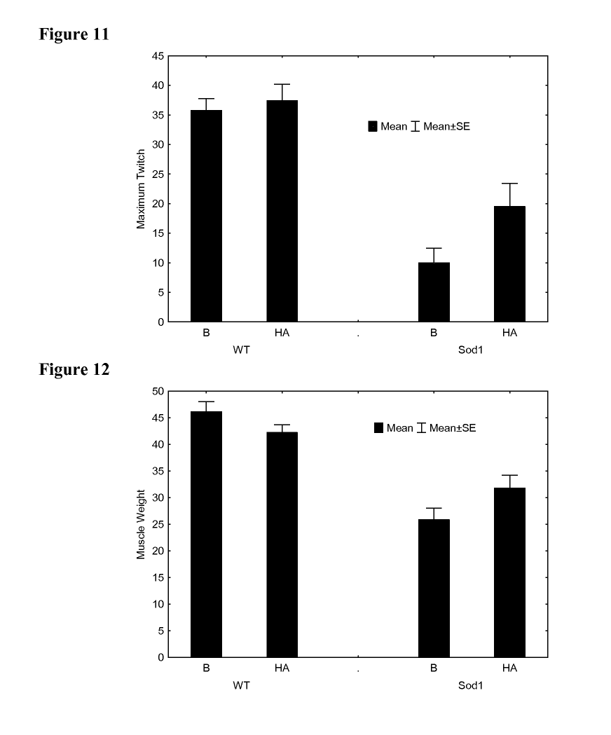

The maximum force generated by the TA muscle at 90 days was significantly reduced in SOD1 mice and this was significantly improved by anti-Nogo-A treatment (p value 0.0314). The results are shown in FIG. 11.

ix. TA Muscle Weight

The weight of the TA muscle shows some reduction at 90 days in SOD1 mice and while there was a treatment-related trend to improvement with anti-Nogo-A this did not reach significance at this stage (p value 0.0578). The results are shown in FIG. 12.

x. TA Time to Peak

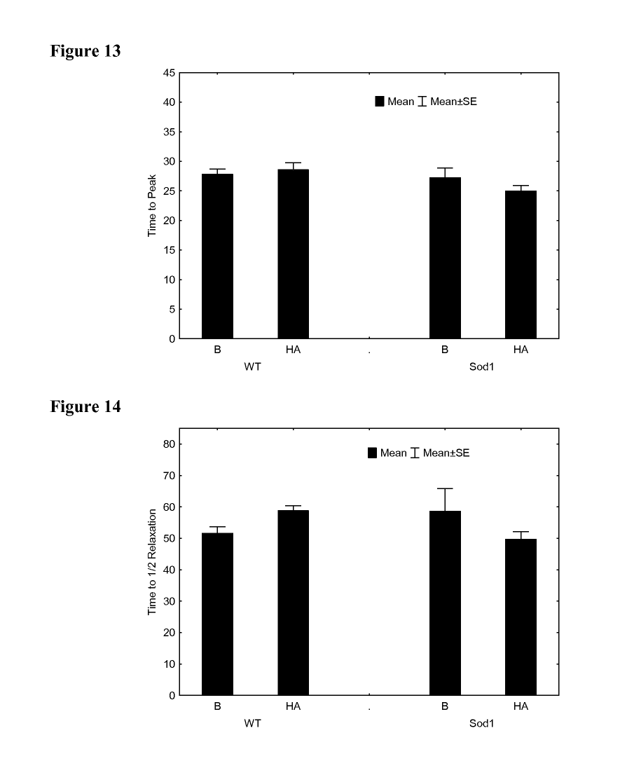

No significant differences were observed between any of the groups in the time to reach peak force generation at this stage (FIG. 13).

xi. TA Time to ½ Relaxation

No significant differences were seen in this measure at this stage in these groups (FIG. 14).

### Example 3

### In Vivo ALS Model Muscle Physiology Studies with 2A10 and Riluzole

Female transgenic SOD1^{G93A} mice (selected as per example 2) were divided into 9 experimental groups consisting of SOD1 and wild type (WT) littermates. Three groups of WT littermates (n=10 unless otherwise indicated) served as controls, and were treated with Phosphate buffered saline (PBS), riluzole alone (30 mg/kg) or anti-Nogo A antibody (2A10, WO2005061544) alone (30 mg/kg).

Six groups of SOD1 mice (n=10 unless otherwise indicated) were treated with anti-Nogo A antibody (2A10, WO2005061544) at two concentrations (3 mg/kg or 30 mg/kg) alone or in combination with Riluzole (30 mg/kg):
Group I: WT treated with PBS (n=5)
Group II: WT treated with Riluzole (30 mg/kg per day)
Group III: WT treated with Anti-Nogo Antibody (30 mg/kg)+Riluzole (30 mg/kg per day)
Group IV: SOD1 treated with PBS (n=5)
Group V: SOD1 treated with Anti-Nogo Antibody (30 mg/kg)
Group VI: SOD1 treated with Anti-Nogo Antibody (3 mg/kg) (n=5)
Group VII: SOD1 treated with Riluzole (30 mg/kg per day)
Group VIII: SOD1 treated with Anti-Nogo Antibody (30 mg/kg)+Riluzole (30 mg/kg per day)
Group IX: SOD1 treated with Anti-Nogo Antibody (3 mg/kg)+Riluzole (30 mg/kg per day)

Riluzole was administered orally in the drinking water from 65 days of age until 90 days of age. The daily dosages were calculated based on a daily water intake of 5 ml. Fresh solutions were prepared once a week with the total consumed volume measured in order to ensure a constant daily and weekly dose. Water intake was monitored and did not differ between the groups and was in the expected range of 5 ml.

Anti-Nogo A antibody or vehicle (PBS) was administered by i.p. injections weekly, starting from 70 days of age until 90 days of age (3 injections).

Disease progression was assessed as per example 2.

### Results

The riluzole-anti-Nogo-A SOD1 study was a large study that aimed to look at a number of parameters across nine treatment groups. This required the use of mice from more litters than usual and will have contributed to additional variability and reduced sensitivity to see beneficial and additive or synergistic treatment effects. To limit the total number of groups required we chose to select the 30 mg/kg dose based on published efficacy in the SOD1 mouse model (Waibel et al 1994) mindful of the fact that this is a high dose and that some aspects of Riluzole pharmacology such as the asthesia (muscle weakness) it can cause may limit the observed combinatorial effects of treatment. In subsequent studies it may be possible to explore additional lower doses of riluzole to extend our current observations, doses which may correlate more closely to the likely exposures seen in ALS patients commonly receiving up to 100 mg/day. Higher doses of riluzole are associated with adverse events including asthesia in patients and these tend to limit the ability of physicians to significantly increase doses. It is therefore plausible that at the lower doses of riluzole utilised in human therapy there may be an even greater opportunity to observe significantly enhanced efficacy of anti-Nogo-A plus riluzole or other glutamate modulating agents.

i. EDL Motor Unit Number Estimation

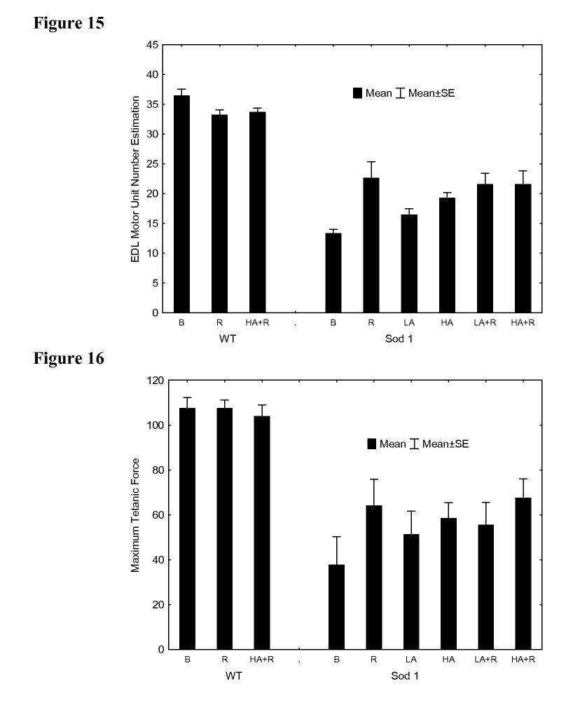

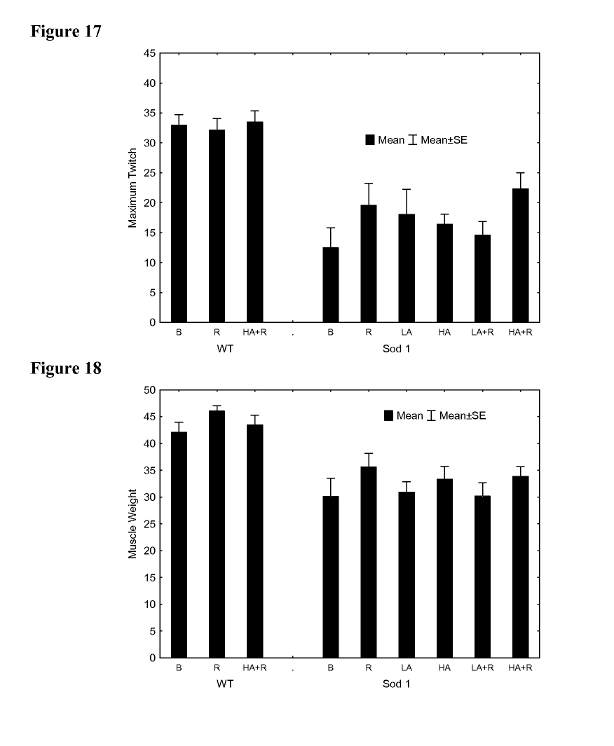

Electrical stimulation of Extensor digitorum longus (EDL) muscle with increasing intensity is able to induce activation of successively greater motor units with each producing a characteristic trace. Summation of the traces can be used to produce an estimate of surviving motor unit numbers. Disease progression in SOD1 mice results in a significant and progressive reduction in motor unit traces. In our previous studies we have found a good correlation between motor unit estimation and direct counts of motor neuron numbers in the spinal cord. Importantly, this determination of motor unit numbers is likely to represent a good correlate of similar clinical determinations such as MUNE (motor unit number estimate). In this study we saw a significant and dose-dependent increase in motor unit numbers in 2A10 treated SOD1 mice (LA 3 mg/kg, HA 30 mg/kg, dosed weekly from day 70). At the high dose of anti-Nogo-A 2A10 the effect was comparable in magnitude with high dose Riluzole (30 mg/kg, dosed in drinking water from day 65). The results are shown in FIG. 15.

Detection of a combinatorial effect between riluzole and 2A10 was made difficult in this test as both Riluzole alone at the 30 mg/kg dose used, and 2A10 HA were clearly efficacious and statistically significantly different from SOD-B vehicle animals.

ii. TA Maximum Tetanic Force

Repetive tetanic electrical stimulation of the mouse Tibialis Anterior (TA) muscle can be used to produce a measure of the maximum force that can be generated by this muscle. Disease progression in the SOD1 mice produces a significant and progressive muscle weakening that is clearly evident at day 90 as shown here (FIG. 16). Such measures of strength have a direct and relevant correlation with the decline in strength seen in ALS patients. Interestingly, Riluzole alone (30 mg/kg) and anti-Nogo-A alone 30 mg/kg each failed to reach statistical significance relative to the SOD1-B vehicle group (p values 0.0607 and 0.1219 respectively) while the combination of the two results in a significant improvement (p value 0.039) that may be indicative of a synergistic effect of Riluzole and anti-Nogo-A treatment (despite the high dose of riluzole used in this study).

iii. TA Maximum Twitch

A single pulsatile electrical stimulation of the TA muscle can be used to measure the force generated during the muscle twitch. Again, as with the maximum tetanic force measure it was interesting that in the SOD1 treated groups only the combination of Riluzole (30 mg/kg) and high anti-Nogo-A (30 mg/kg) reached statistical significance relative to the SOD 1-vehicle group (p value 0.0199), suggestive of an additive or synergistic effect of the two treatments.

iv. TA Muscle Weight

Disease progression in SOD1 mice is associated with a reduction in muscle weight that is very apparent at late stages of disease (>120 days) but can also be detected at earlier times including day 90 as shown here. While there was a small but significant effect of genotype on muscle weight none of the treatments reached significance compared with the vehicle SOD1 group (FIG. 18).

v. TA Time to Peak

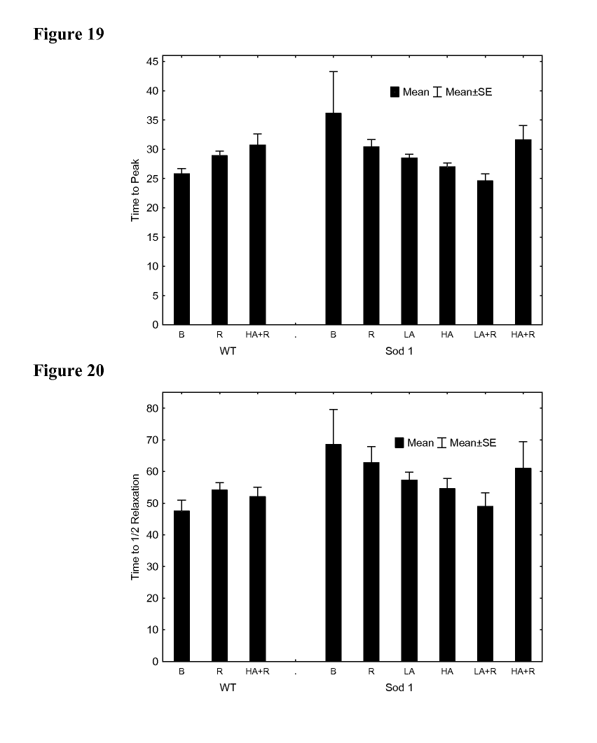

During electrical stimulation of the TA muscle one of the parameters that can be measured is the time taken to reach the peak of force generation. The significance of this measure is unclear and actually showed no statistical difference between wild-type and SOD1 vehicle groups (FIG. 19). It is therefore difficult to interpret the small changes seen in this measure at this time-point.

vi. TA Time to ½ Relaxation

The contraction and subsequent relaxation of the TA muscle following electrical stimulation can be measured and disease progression at later stages produces a dramatic effect on this measure. At day 90 as shown here (FIG. 20) the effects were more limited with the small difference between WT and SOD1 vehicle groups (p value 0.0205). While there is a trend to normalisation with Riluzole and the anti-Nogo-A groups it is only the Riluzole plus 3 mg/kg anti-Nogo-A that reaches statistical significance (p value 0.0212). The limited dynamic range of the assay at this time-point limits further interpretation of this data at this stage.

1. A method for the treatment or prophylaxis of amyotrophic lateral sclerosis, comprising administering to a patient in need thereof a therapeutically effective amount of a Nogo-A antagonist.

2. A method according to claim 1, which further comprises administering a therapeutically effective amount of at least one compound with anti-glutamate activity to the patient.

3. A method according to claim 2, wherein said at least one compound with anti-glutamate activity is riluzole.

4. A method according to claim 1, wherein the Nogo-A antagonist is an anti-Nogo-A antibody.

5. A method according to claim 4, wherein said Nogo-A antagonist is a humanised or human antibody.

6. A method according to claim 5, wherein said anti-Nogo-A antibody is one of H20L16, H28L16, H28L13 and H27L16.

7. A method according to claim 5, wherein said Nogo-A antibody is H28L16 (SEQ ID NO:2 and SEQ ID NO:4).

8. A method according claim 3, wherein the Nogo-A antagonist and the riluzole are co-administered to the patient.

9. A method according to claim 8, wherein about 50 mg to about 150 mg riluzole is administered to the patient daily.

10. A method according to claim 8, wherein 100 mg riluzole is administered to the patient daily.

11. A method according to claim 8, wherein the riluzole is orally administered.

12. A method according to claim 1, wherein about 2 mg/kg to 40 mg/kg of Nogo-A antagonist is administered to the patient.

13. A method according to claim 1, wherein the Nogo-A antagonist is administered intravenously to the patient.

14. A method according to claim 1, wherein said Nogo-A antagonist is administered to the patient weekly.

15. A method according to claim 1, wherein said Nogo-A antagonist is administered to the patient once every two weeks.

16. A method according to claim 1, wherein said Nogo-A antagonist is administered to the patient once every four weeks.

17. A method according to claim 1, wherein the patient has failed to respond, or has shown an inadequate response, to the use of riluzole in the treatment or prophylaxis of ALS.

18. A Nogo-A antagonist for use in the treatment or prophylaxis of amyotrophic lateral sclerosis.

19. A Nogo-A antagonist according to claim 18, wherein the Nogo-A antagonist is co-administered to the patient with a compound having anti-glutamate activity.

20. Use of a Nogo-A antagonist in the manufacture of a medicament for the treatment or prophylaxis of amyotrophic lateral sclerosis.

21. Use according to claim 20, wherein the Nogo-A antagonist is co-administered with a compound having anti-glutamate activity for the treatment or prophylaxis of amyotrophic lateral sclerosis.

22. A pharmaceutical composition comprising at least one Nogo-A antibody.

23. The pharmaceutical composition of claim 22, wherein said Nogo-A antibody is H28L16 (SEQ ID NO:2 and SEQ ID NO:4), H28L13 (SEQ ID NO:2 and SEQ ID NO:3) and H27L16 (SEQ ID NO:1 and SEQ ID NO:4).

24. The pharmaceutical composition of claim 22, further comprising at least one compound with anti-glutamate activity.

25. A kit-of-parts comprising at least one anti-Nogo-A antibody and at least one compound with anti-glutamate activity.

26. The pharmaceutical composition of claim 24 or kit-of-parts of claim 25, wherein said compound having anti-glutamate activity is riluzole.

27. A method according to claim 2, wherein the Nogo-A antagonist and at least one compound with anti-glutamate activity have a synergistic effect when co-administered to said patient.
